(19) United States
(12) Patent Application Publication
(10) Pub. No.: US 2007/0292521 A1
(43) Pub. Date: Dec. 20, 2007

(54) Oral Encapsulated Preparation for Aquatic Animals

(75) Inventors: Tsun-Yung Kuo, I-Lan (TW); Gabriel Hsu-Chung Chen, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: SCHWITZER CO., LTD, Taipei (TW)

(21) Appl. No.: 11/425,143

(22) Filed: Jun. 20, 2006

(51) Int. Cl. A61K 36/02; A61K 35/12; A61K 9/50

(52) U.S. Cl. 424/490; 424/195.17; 424/520

(57) ABSTRACT

An oral encapsulated preparation for aquatic animals, is an water/oil/water type encapsulated preparation which comprises on a weight basis: 50 to 80% of aqueous phase containing water-soluble active agent; 20 to 50% of oily phase containing one or more types of oil; 1 to 5% of the first emulsifying agent; 1 to 5% of the second emulsifying agent. In preparation, an oily material is mixed thoroughly with the first emulsifying agent to create a first mixture, then a portion of an aqueous material is added to the first mixture followed by fast shaking/agitation to create a second mixture; the unused, remaining portion of the aqueous material is then mixed thoroughly with the second emulsifying agent and is added to the second mixture followed by shaking/aggitation to prepare the oral encapsulated preparation of this invention.

### BACKGROUND OF THE INVENTION

1. Field of the Invention

This invention features oral encapsulated preparations that especially indicate fine-particle, highly stable, appetizing preparations used as an oral vaccine and as various supplements (such as nutrient, immune enhancing agent or booster, pathogen eradicator and so on) for aquatic animals.

2. Description of the Prior Art

The technology for aquatic farming is well developed in Taiwan, which is known as one of top three largest aquatic farming nations, together with Japan and Norway. However, subtropical climate and intensive aquatic farming in Taiwan facilitate transmission of pathogens in aqueous environment. In addition, intensive aquatic farming requires high quality of nutrient and farming environment to prevent high mortality rate of aquatic animals. Such potentially high mortality rate can tremendously impact the economy and fishermen's interests.

Aquatic vaccination is usually applied at the stage of fingerling or juvenile prawn. There are three types of administration: injection, immersion and orally administration; wherein injection costs time, labor and easily causes stress to susceptible fish and shrimp. Immersion is the easiest among the three, but not very suitable for every kind of vaccine. Moreover, the amount of antigen or antibody to be applied is often not definite for immersion, and typically needs to be tested on target fingerling in the field. As for oral vaccine, it is typically made by adding vaccine to bait, and subsequently applying them together to aquatic animals. This is typically the most cost-effective way of administration. The amount to be applied is less than that of immersion, and can reach satisfactory level of effectiveness. As a result, oral vaccine has been a major focus in current vaccine development for aquatic animals.

Yang, et al. discloses an oral vaccine for aquatic animals in ROC (TAIWAN) patent No. 1227114 (referred to as cited reference below and incorporated herein by reference in its entirety). This vaccine includes multi-cellular organisms as food for aquatic animals, and also includes transformed single-cell organisms that express the recombinant antigen stimulating immune response or anti-disease substance production in aquatic animals. Moreover, the cited reference also discloses a way of orally applying the vaccine with the concept of bioencapsulation, wherein the transformed single-cell organism expressing recombinant antigen is encapsulated in multi-cellular organisms which is used as feed for vaccinating aquatic animals.

However, the vaccine used in the cited reference is produced by transforming the single-cell organism with recombinant gene, and the stability and the expression of the recombinant gene can effect the expression of the antigen. Therefore, this way of preparation is not suitable for all vaccines. Although orally administrating vaccine is relatively convenient and has the least negative impact on target aquatic animals, the method disclosed by Yang et. al. has not been popular because development of recombinant gene and transforming technology is relatively time-consuming and expensive, and the amount of gene expression is not stable.

U.S. Pat. No. 5,424,067 (referred to as cited reference below and incorporated herein by reference in its entirety) describes injectable multi-phase solutions of W/O/W type. This vaccine of the W/O/W type is defined on a weight basis and contains: (1) 20-78% of an aqueous phase containing one or more antigens; (2) 2-10% of an emulsifying system comprising one or more non-ionic, non-toxic emulsifiers with inversion point between 25-45° C.; and (3) 20-70% of a water immiscible oily material containing one or more oils selected from the groups consisting of: (a) mineral oil or synthetic hydrocarbons which is liquid at 4° C. and have a viscosity lower than 100 mPas at 40° C.; (b) synthetic oils having at least 14 carbon atoms; (c) oils of vegetable origin; (d) oils of animal origin (especially squalene). The method of preparing the W/O/W type vaccine is to place oily material (including oil and emulsifying agents) and aqueous material (containing antigen or other active agent) at the same temperature (between 20-40° C.), normally at 30° C., then the aqueous material is gently poured into the oily material, followed by tenderly mixing the resulting mixture by stirring until its temperature reaches room temperature for usage.

The W/O/W type vaccine of the cited reference has low viscosity and high fluidity, and is appropriate for use as injectable vaccine for livestock. The effectiveness of the vaccine is similar to Freund's incomplete adjuvant (FIA). However, due to the low stability and difficult production process of this W/O/W type vaccine, its usage is limited as injectable vaccine for livestock, and is undesirable for aquatic animals and oral vaccine preparation.

Therefore, while various oral preparations for aquatic animals are known in the art, all of most of them suffer from one or more disadvantages. Thus, there remains a considerable need for oral preparation for aquatic animals.

Based on the defects described above, the inventor of the instant application took several years of research to successfully invent an oral encapsulated preparation for aquatic animals, which will be described in detail below.

### SUMMARY OF THE INVENTION

The first purpose of this invention is to provide a fine-particle, highly stable, and appetizing oral encapsulated preparation.

The second purpose of this invention is to provide an encapsulated preparation that can be used as oral vaccines and various supplements.

The third purpose of this invention is to provide a method of administrating material orally.

The oral encapsulated preparation for aquatic animals that meets the above purposes is an encapsulated preparation of W/O/W type, which includes:

an aqueous phase containing an aqueous material, water-soluble active agent and representing between 50 and 80% of the formula;

an oily phase containing one or more type of oily material and representing between 20 and 50% of the formula;

a first emulsifying agent representing between 1 and 5% of the formula;

a second emulsifying agent representing between 1 and 5% of the formula.

The oily material is mixed well with the first emulsifying agent to create a first mixture, then, a portion of the aqueous material is added into the first mixture to create a second mixture by fast shaking/aggitation. The unused, remaining portion of aqueous material is mixed thoroughly with the second emulsifying agent to create a third mixture and this third mixture is added to the second mixture followed by fast shaking/aggitation to create a fourth mixture. The oral encapsulated preparation of this invention is made, as represented by the fourth mixture.

The water-soluble active agent of the encapsulated preparation of this invention could be antigen, antibody, antibiotic, nutrient, immune enhancing agent or booster, pathogen eradicator and so on.

The oils of the encapsulated preparation of this invention could be one or more members selected from the following group: (a) mineral oil or synthetic hydrocarbons which is liquid at 4° C. and have a viscosity lower than 100 mPas at 40° C.; (b) synthetic oils having at least 14 carbon atoms; (c) oils of vegetable origin; and (d) oils of animal origin.

The first emulsifying agent of the encapsulated preparation of this invention includes one or more non-ionic, non-toxic emulsifying agents of which the inversion point is between 25-45° C., and is selected from one or more members of the following group: sorbitan fatty acid ester; concentrate of sorbitan fatty acid ester and ethylene oxide or propylene oxide; mannitol fatty acid ester; concentrate of mannitol fatty acid ester and ethylene oxide or propylene oxide; compound of mannitol fatty acid ester and one selected from the hydrophilic group: carboxylic acid, amine, amide, alcohol, polyol, ether, oxide; anhydromannitol fatty acid ester; compound of anhydromannitol fatty acid ester and one selected from the hydrophilic group: carboxylic acid, amine, amide, alcohol, polyol, ether, oxide; saccharose fatty acid ester; concentrate of saccharose fatty acid ester and ethylene oxide or propylene oxide; glycerine fatty acid ester; concentrate of glycerine fatty acid ester and ethylene oxide or propylene oxide; concentrate of fatty acid and ethylene oxide or propylene oxide; concentrate of fatty alcohol and ethylene oxide or propylene oxide; and glycerophospholipid.

The second emulsifying agent of the encapsulated preparation of this invention includes one or more non-ionic, non-toxic emulsifying agents of which the inversion point is between 25-45° C., and is selected from one or more members of the following group: sorbitan fatty acid ester; concentrate of sorbitan fatty acid ester and ethylene oxide or propylene oxide; mannitol fatty acid ester; concentrate of mannitol fatty acid ester and ethylene oxide or propylene oxide; compound of mannitol fatty acid ester and one selected from the hydrophilic group: carboxylic acid, amine, amide, alcohol, polyol, ether, oxide; anhydromannitol fatty acid ester; compound of anhydromannitol fatty acid ester and one selected from the hydrophilic group: carboxylic acid, amine, amide, alcohol, polyol, ether, oxide; saccharose fatty acid ester; concentrate of saccharose fatty acid ester and ethylene oxide or propylene oxide; glycerine fatty acid ester; concentrate of glycerine fatty acid ester and ethylene oxide or propylene oxide; concentrate of fatty acid and ethylene oxide or propylene oxide; concentrate of fatty alcohol and ethylene oxide or propylene oxide; and glycerophospholipid.

This invention further provides a method of administrating material orally by using the concept of bioencapsulation. Firstly, the encapsulated preparation of this invention is used to feed multi-cellular organism, and the multi-cellular organism is then used as food for target aquatic animals. This effectively helps the intake of encapsulated preparation in large quantities by target aquatic animals.

The multi-cellular organisms are biological bait selected from one or more members of the following group including Artema Salina L., Rotifera, Copepoda, algae and Paramecium. The target aquatic animals can be fish and shrimp. Many other aquatic and non-aquatic animals are also contemplated as target animals.

This invention further provides a method of administrating material orally by adding the contemplated oral encapsulated preparation for aquatic animals to the animal feed. This method effectively helps the intake of encapsulated preparation in large quantities by target aquatic animals.

Contemplated method includes adding the oral encapsulated preparation for aquatic animal to the feed by coating the preparation on the outer layer of the feed.

Another contemplated method includes adding the oral encapsulated preparation for aquatic animal to the feed by mixing the preparation with the feed.

Contemplated target aquatic animals include fish and shrimp.

These features and advantages of the present invention will be fully understood and appreciated from the following detailed description of the accompanying drawings.

### BRIEF DESCRIPTION OF THE DRAWINGS

FIG. 1 displays the oral vaccine of the oral encapsulated preparation for aquatic animals of this invention observed under microscope.

FIG. 2 shows Artema Salina L. after being fed with the oral vaccine of this invention for aquatic animals as it is observed under microscope.

FIG. 3A displays the mean survival rate of the juvenile shrimp in Control and Vaccinated group of example 2.

FIG. 3B displays the individual body length of the juvenile shrimp in Control and Vaccinated group of example 2.

FIG. 3C displays the individual body weight of the juvenile shrimp in Control and Vaccinated group of example 2.

FIG. 3D displays the length/weight ratio of the juvenile shrimp in Control and Vaccinated group of example 2.

FIG. 3E displays the guts to muscle ratio of the juvenile shrimp in Control and Vaccinated group of example 2.

FIG. 3F displays the mean survival rate of the juvenile shrimp after twenty-four hour stress test in Control and Vaccinated group of example 2.

FIG. 4A displays the mean survival rate of the juvenile shrimp in Control and Vaccinated group of example 3.

FIG. 4B displays the individual body length of the juvenile shrimp in Control and Vaccinated group of example 3.

FIG. 4C displays the individual body weight of the juvenile shrimp in Control and Vaccinated group of example 3.

FIG. 4D displays the length/weight ratio of the juvenile shrimp in Control and Vaccinated group of example 3.

FIG. 4E displays the guts to muscle ratio of the juvenile shrimp in Control and Vaccinated group of example 3.

FIG. 4F displays the mean survival rate of the juvenile shrimp after twenty-four hour stress test in Control and Vaccinated group of example 3.

### DETAILED DESCRIPTION OF THE PREFERRED EMBODIMENT

### EXAMPLE 1

### The Preparation of the Oral Vaccine for Aquatic Animal

The material encapsulated in this example is vibriosis vaccine and preparation of the oral vaccine of this example is according to the following formula:

An aqueous material containing vibriosis vaccine and representing 65% of the formula;

an oil material representing 30% of the formula;

a first emulsifying agent representing 2.5% of the formula;

a second emulsifying agent representing 2.5% of the formula.

After thoroughly mixing the oil material and the first emulsifying agent to create a first mixture, 50% of the aqueous material is added to the first mixture to create a second mixture, followed by fast shaking/aggitation at 3000 rpm to mix the second mixture. Then the remaining 50% of the unmixed aqueous material and the second emulsifying agent are thoroughly mixed, and the resulting mixture is then added to the second mixture. After fast shaking/aggitation at 3000 rpm, the oral vaccine of this example is made, and:

wherein the oil material is fish oil;

wherein the first emulsifying agent is sorbitan fatty acid ester; concentrate of sorbitan fatty acid ester and ethylene oxide or propylene oxide;

wherein the second emulsifying agent is sorbitan fatty acid ester; concentrate of sorbitan fatty acid ester and ethylene oxide or propylene oxide;

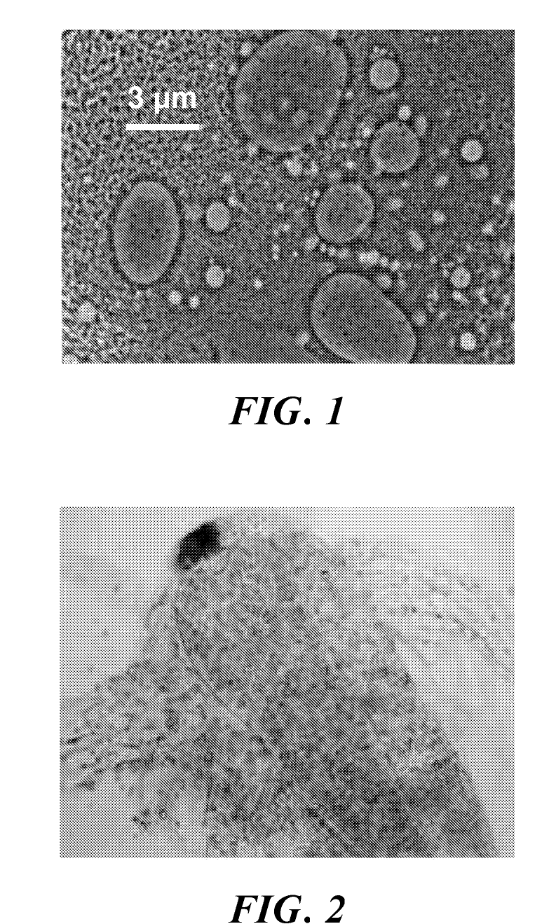

The final product is stored in dark between 4 and 8° C. As displayed in FIG. 1, the oral vaccine for aquatic animals of this invention was observed under the microscope. After treatment of encapsulation technology of this invention, the size of the particle is about or under 10 μm.

### EXAMPLE 2

### The Growth Test of the LiptopEnaeus Vannamei Larvae

Animal to be tested: the larvae of Liptopenaeus vannamei that is specifically pathogen free.

Vaccine to be tested: Oral vaccine prepared in example 1 which is diluted with 1 liter of filtered sea water, is used for vaccination.

Biological bait: The Artemia nauplii are used as orally applied system for vaccination.

Vaccinated group: The Artemia nauplii (0.125 kg/1 liter) are fed with oral vaccine prepared in example 1 for two hours, and are used as biological bait three times a day for Liptopenaeus vannamei (150 larva/1 liter) from stage of post-larvae 4 till post-larvae 13.

Control group: The larva of Liptopenaeus vannamei without vaccination was used as Control group.

As displayed in FIG. 2, Artemia nauplii fed with oral vaccine prepared in example 1 are observed under microscope, and a relatively large amount of oral vaccine was found in the body of Artemia nauplii.

Vaccinated and Control group were proceeded triplicate, and the development condition of Liptopenaeus vannamei larva was observed after post-larvae 14 which includes:

1. Mean survival: the mean survival rate of the triplicate groups was calculated.

2. Individual body length: the body length was calculated using a graticule and microscope, and 100 individuals were measured from the rostral tip to the telson base in each treatment group.

3. Individual body weight: A sample of 2 grams biomass from each treatment group was drained and weighed on blotting paper. Individual post-larvae within the 2 grams sample were then counted to calculate individual body weight.

4. Length/weight ratio: Length/weight ratio was calculated from data derived from points 2 and 3.

5. Gut to muscle ratio: Gut/muscle ratio was calculated using a graticule and microscope. 100 animals were sampled per treatment group. Analysis was conducted on the first abdominal segment.

6. Twenty-four hour stress test: A sample of 100 animals per treatment group was exposed to 500 ppm formalin. Survival was calculated after a 24-hour period.

Results:

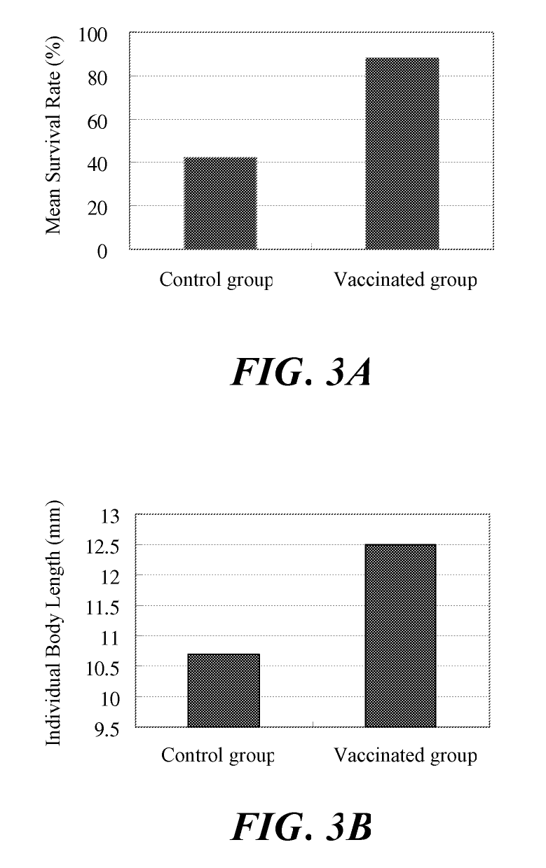

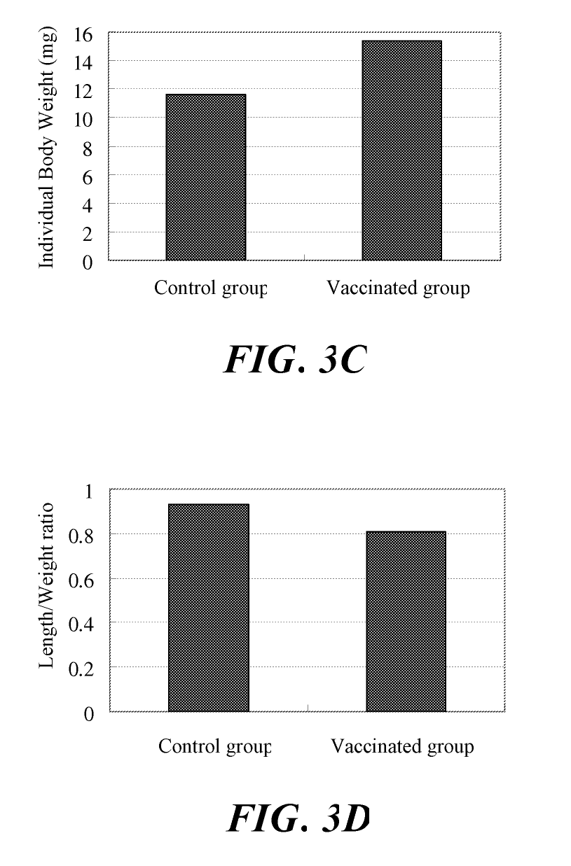

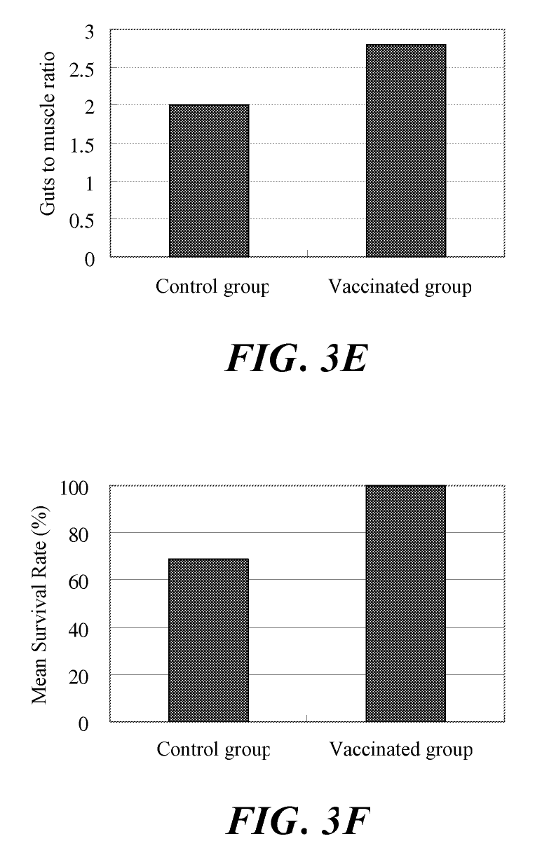

1. Mean survival: as displayed in FIG. 3A, mean survival in Control group was 42.0%, compared to 88.0% of Vaccinated group.

2. Individual body length: as displayed in FIG. 3B, individual body length in Control group was 10.7 mm, compared to 12.5 mm of Vaccinated group.

3. Individual body weight: as displayed in FIG. 3C, individual body weight in Control group was 11.6 mg, compared to 15.4 mg of Vaccinated group.

4. Length/weight ratio: as displayed in FIG. 3D, length/weight ratio in Control group was 0.93, compared to 0.81 of Vaccinated group.

5. Gut to muscle ratio: as displayed in FIG. 3E, Gut to muscle ratio in Control group was 2.0, compared to 2.8 of Vaccinated group.

6. Twenty-four hour stress test: as displayed in FIG. 3F, the mean survival in Control group was 69.0%, compared to 100% of Vaccinated group.

From analysis of results above, the growth of Liptopenaeus vannamei larva after feeding with bio-encapsulated oral vaccine of this invention is far better than that of not vaccinated; not only the length and weight of the larva of Vaccinated group is better than that of Control group, but also gut/muscle ratio and 24-hour stress test of the larva of Vaccinated group is better than that of Control group.

### EXAMPLE 3

### The Growth Test of the Black Tiger Prawn (Pan aeus mon odon) Larvae

Animal to be tested: the larvae of black tiger prawn that is specifically pathogen free.

Vaccine to be tested: the same as example 2.

Biological bait: The Artemia nauplii are used as oral applied system for vaccination.

Vaccinated group: The Artemia nauplii (0.125 kg/1 liter) were fed with oral vaccine prepared in example 1 for two hours, and were used as biological bait three times a day for black tiger prawn (150 larva/1 liter) from stage of post-larvae 4 till post-larvae 13.

Control group: The same batch larva of black tiger prawn without vaccination was used as Control group.

Vaccinated and Control group were proceeded triplicate, and the development of black tiger prawn larva were observed after post-larvae 14, as described in example 2.

Results:

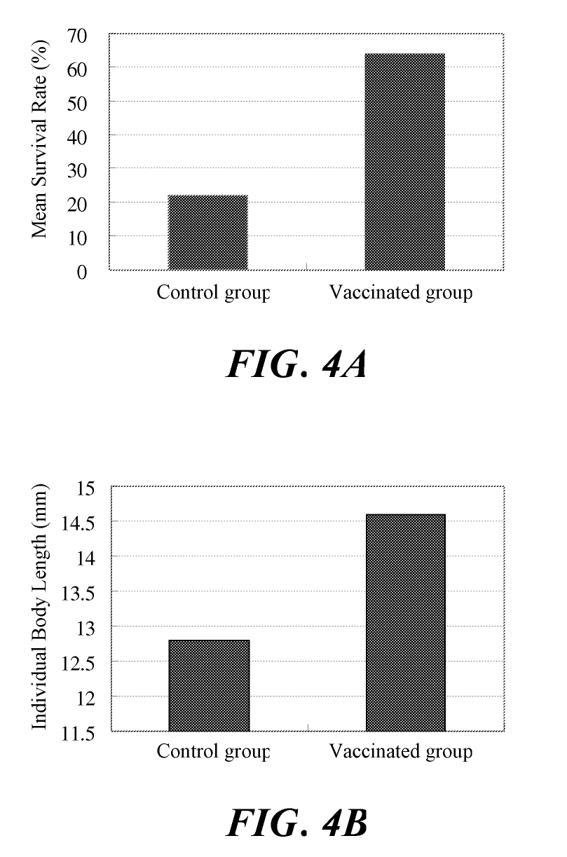

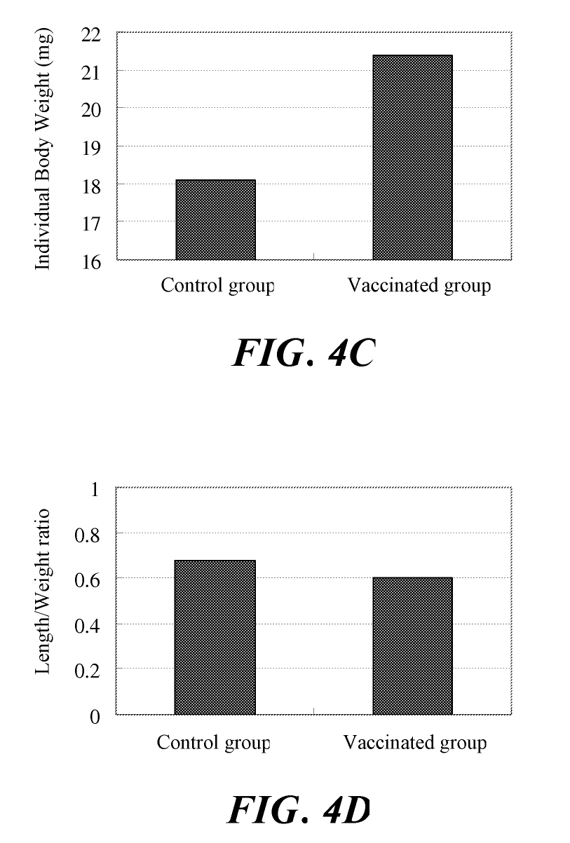

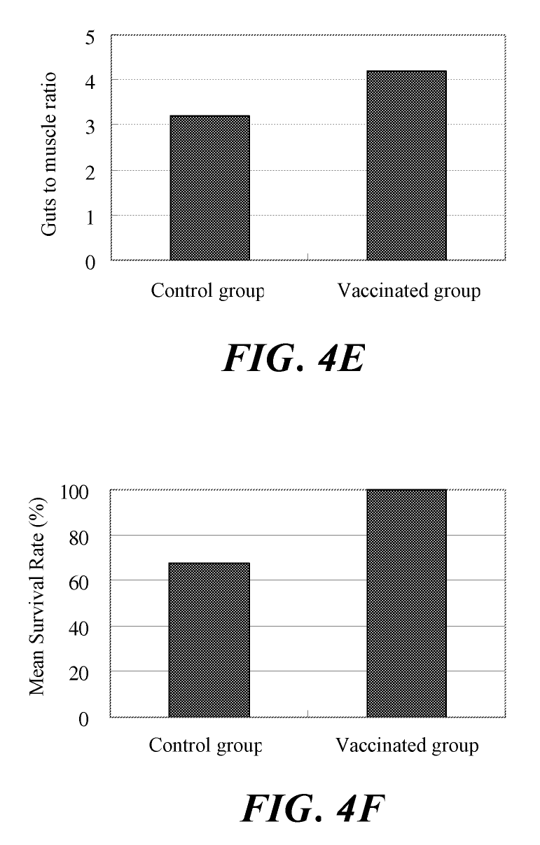

1. Mean survival: as displayed in FIG. 4A, mean survival in Control group was 22.0%, compared to 64.0% of Vaccinated group.

2. Individual body length: as displayed in FIG. 4B, individual body length in Control group was 12.8 mm, compared to 14.6 mm of Vaccinated group.

3. Individual body weight: as displayed in FIG. 4C, individual body weight in Control group was 18.1 mg, compared to 21.4 mg of Vaccinated group.

4. Length/weight ratio: as displayed in FIG. 4D, length/weight ratio in Control group was 0.68, compared to 0.6 of Vaccinated group.

5. Gut to muscle ratio: as displayed in FIG. 4E, gut to muscle ratio in Control group was 3.2, compared to 4.2 of Vaccinated group.

6. Twenty-four hour stress test: as displayed in FIG. 4F, the mean survival in Control group was 68.0%, compared to 100% of Vaccinated group.

From analysis of results above, the growth of black tiger prawn larva after feeding with bio-encapsulated oral vaccine of this invention is far better than that of not vaccinated, not only the length and weight of the larva of Vaccinated group is better than that of Control group, but also gut/muscle ratio and 24-hour stress test of the larva of Vaccinated group is better than that of Control group.

The oral encapsulated preparation for aquatic animals of this invention has advantages listed below when comparing with the cited references described previously and when comparing with other known technology:

1. The W/O/W type of the oral encapsulated preparation for aquatic animals of this invention has relatively low viscosity, produces antibody at a faster rate than that of the known W/O type preparation, and can be used with other drugs.

2. The size of the oral encapsulated preparation for aquatic animals of this invention is less than 100 μm, which is easily eaten by biological bait then the biological bait was used to feed the aquatic animals. Therefore, aquatic animals could take in the oral preparation of this invention in large quantities, which raises absorption rate of the oral encapsulated preparation.

3. Current vaccines and various materials could be used as the materials to be encapsulated in oral encapsulated preparation for aquatic animals of this invention.

4. The oral encapsulated preparation for aquatic animals of this invention was made with fish oil, which contains rich non-saturated fatty acid Ω-3 (omega-3), EPA (Elcosapentaenoic acid) and DNA (Docosahexaenoic acid). So the preparation could be the nutrient for aquatic animals.

5. The appetizing characteristic of fish oil is attractive to biological bait, so the oral encapsulated preparation for aquatic animals of this invention is not only used as the interface for encapsulation and nutrient, but is also used as attractive agent to biological bait.

The description above is just one of the embodiments of this invention. It should be apparent and understood, however, to those skilled in the art that the above embodiment should not be taken as a restriction of this invention. And, that many more modifications besides those already described are possible without departing from the inventive concepts herein. The equipotent embodiment or any change that does not depart from the art of this invention, such as different encapsulated materials, different aqueous material and oily material ratio, the type of oil used and type of emulsifying agent used and the other equipotent embodiments should be included in the patent of this invention. Furthermore, where a definition or use of a term in a reference, which is incorporated by reference herein is inconsistent or contrary to the definition of that term provided herein, the definition of that term provided herein applies and the definition of that term in the reference does not apply.

Based on the description above, this case is not only an invention, but also improves upon known preparation and provides the advantages as described, which fully qualifies for innovation and improvement essentials of invention patent application.

Many changes and modifications in the above-described example of the invention can, of course, be carried out without departing from the scope thereof. Accordingly, to promote the progress in science and the useful arts, the invention is disclosed and intends to be limited only by the scope of the appended claims.

What is claimed is:

1. An oral encapsulated preparation for aquatic animals include:
an aqueous phase containing water-soluble active agent and representing between 50 and 80% of the formula;
an oily phase material containing one or more types of oil and representing between 20 and 50% of the formula;
the first emulsifying agent representing between 1 and 5% of the formula; and
the second emulsifying agent representing between 1 and 5% of the formula.

2. The oral encapsulated preparation for aquatic animals as claim 1, wherein the said water-soluble active agent could be antibody, antigen, antibiotic, nutrient, immune enhancing agent or booster, pathogen eradicator and so on.

3. The oral encapsulated preparation for aquatic animals as claim 1, wherein the said oil material includes one or more selected from this group: mineral oil or synthetic hydrocarbons which is liquid at 4° C. and have a viscosity lower than 100 mPas at 40° C., synthetic oils having at least 14 carbon atoms, oils of vegetable origin, oils of animal origin.

4. The oral encapsulated preparation for aquatic animals as claim 3, wherein the said mineral oil contains more than sixteen carbons long chain and are not aromatic compounds.

5. The oral encapsulated preparation for aquatic animals as claim 3, wherein the said synthetic hydrocarbon compound is polyisobutene or polyisoprene.

6. The oral encapsulated preparation for aquatic animals as claim 3, wherein the said vegetable oil is non-saturated and biodegradable.

7. The oral encapsulated preparation for aquatic animals as claim 3, wherein the said animal oil is fish oil.

8. The oral encapsulated preparation for aquatic animals as claim 1, wherein the said the first emulsifying agent contains one or more nonionic, nontoxic emulsifying agent and has inversion point between 25-45° C.

9. The oral encapsulated preparation for aquatic animals as claim 8, wherein the first emulsifying agent includes one or more non-ionic, non-toxic emulsifying agent, has inversion point between 25-45° C., and is selected from one or more of the group: sorbitan fatty acid ester; concentrate of sorbitan fatty acid ester and ethylene oxide or propylene oxide; mannitol fatty acid ester; concentrate of mannitol fatty acid ester and ethylene oxide or propylene oxide; compound of mannitol fatty acid ester and one selected from the hydrophilic group: carboxylic acid, amine, amide, alcohol, polyol, ether, oxide; anhydromannitol fatty acid ester; compound of anhydromannitol fatty acid ester and one selected from the hydrophilic group: carboxylic acid, amine, amide, alcohol, polyol, ether, oxide; saccharose fatty acid ester; concentrate of saccharose fatty acid ester and ethylene oxide or propylene oxide; glycerine fatty acid ester; concentrate of glycerine fatty acid ester and ethylene oxide or propylene oxide; concentrate of fatty acid and ethylene oxide or propylene oxide; concentrate of fatty alcohol and ethylene oxide or propylene oxide; and glycerophospholipid.

10. The oral encapsulated preparation for aquatic animals as claim 1, wherein said the second emulsifying agent contains one or more nonionic, nontoxic emulsifying agent and the inversion point is between 25-45° C. of said the second emulsifying agent.

11. The oral encapsulated preparation for aquatic animals as claim 10, wherein said the second emulsifying agent includes one or more non-ionic, non-toxic emulsifying agent of which the inversion point is between 25-45° C., and is selected from one or more of the group: sorbitan fatty acid ester; concentrate of sorbitan fatty acid ester and ethylene oxide or propylene oxide; mannitol fatty acid ester; concentrate of mannitol fatty acid ester and ethylene oxide or propylene oxide; compound of mannitol fatty acid ester and one selected from the hydrophilic group: carboxylic acid, amine, amide, alcohol, polyol, ether, oxide; anhydromannitol fatty acid ester; compound of anhydromannitol fatty acid ester and one selected from the hydrophilic group: carboxylic acid, amine, amide, alcohol, polyol, ether, oxide; saccharose fatty acid ester; concentrate of saccharose fatty acid ester and ethylene oxide or propylene oxide; glycerine fatty acid ester; concentrate of glycerine fatty acid ester and ethylene oxide or propylene oxide; concentrate of fatty acid and ethylene oxide or propylene oxide; concentrate of fatty alcohol and ethylene oxide or propylene oxide; and glycerophospholipid.

12. The method to prepare the oral encapsulated preparation for aquatic animals as claim 1, which includes the steps below:
Step 1 said oily phase material is mixed thoroughly with said first emulsifying agent;
Step 2 adding said aqueous phase material to the mixture in step 1 and mixing them by fast shaking;
Step 3 said aqueous phase material left is mixed with said second emulsifying agent, then is added to said mixture in step 2 and mixed by fast shaking.

13. The method to prepare the oral encapsulated preparation for aquatic animals as claim 12, wherein the fast shaking speed in step 2 is 1000-7000 rpm.

14. The method to prepare the oral encapsulated preparation for aquatic animals as claim 12, wherein the fast shaking speed in step 3 is 1000-7000 rpm.

15. A method for orally delivering material, which uses said oral encapsulated preparation for aquatic animals as claim 1 to feed multi-cellular organism, and the multi-cellular organism is fed to target aquatic animal, making target aquatic animals effectively intake said encapsulated preparation with large quantities.

16. A method for orally delivering material as claim 15, wherein said multi-cellular organism is biological bait selected from Artema Salina L., Rotifera, Copepoda, algae and Paramecium.

17. A method for orally delivering material as claim 15, wherein said target aquatic animals include fish and shrimp.

18. A method for orally delivering material, which adds said oral encapsulated preparation for aquatic animals as claim 1 into the feed, and then the feed was used to nourish target aquatic animals, letting target aquatic animals effectively intake said encapsulated preparation with large quantities.

19. A method for orally delivering material as claim 18, wherein said oral encapsulated preparation for aquatic animals is made by coating on the outer layer of the feed.

20. A method for orally delivering material as claim 18, wherein the said oral encapsulated preparation for aquatic animals is made by mixing with the feed.

21. A method for orally delivering material as claim 18, wherein said target aquatic animals include fish and shrimp.
